(19) United States
(12) Patent Application Publication
(10) Pub. No.: US 2009/0024149 A1
(43) Pub. Date: Jan. 22, 2009

(54) RIVET INTRODUCTION SYSTEM

(75) Inventors: Zahid A. Saeed, Cincinnati, OH (US); Vihar C. Surti, Winston-Salem, NC (US); Kenneth C. Kennedy, Clemmons, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/173,343

(22) Filed: Jul. 15, 2008

Related U.S. Application Data
(60) Provisional application No. 60/950,209, filed on Jul. 17, 2007.

(51) Int. Cl. A61B 17/08; A61B 17/04

(52) U.S. Cl. 606/151; 606/213

(57) ABSTRACT

A rivet, rivet introduction device, and method therefor. The rivet is configured for deployment from a rivet introduction device to provide for connection of tissue. The rivet preferably includes two expansion zones configured to secure tissue therebetween, with the expansion zones being either self-expanding or expandable by use of a mandrel. The rivet introduction device preferably is configured to provide for introduction of a rivet with access only to one side of the tissue to be connected.

### CROSS-REFERENCE TO RELATED APPLICATIONS

This application claims priority to U.S. Provisional Application Ser. No. 60/950,209, filed Jul. 17, 2007, which is incorporated herein by reference in its entirety.

### FIELD OF THE INVENTION

The present invention relates to a medical device for use in endoscopic surgery, and more particularly, to a device and system configured to introduce rivets for use in endoscopic or laparoscopic tissue repair.

### BACKGROUND

Tissue repair during endoscopic and laparoscopic procedures presents several difficulties. Given the size and mobility constraints associated with these minimally invasive surgical methods, there are special challenges to traditional tissue repair methods such as suturing. This problem has prompted the development of a number of endoscopic therapeutic approaches including the use of sutures, staples, clips, and similar devices. Although such approaches are often effective, each requires use of specialized equipment and techniques requiring accessory tools and/or particular dexterity. Open surgery to repair tissue injuries such as perforations or tears to gastric or intestinal tissues require invasive techniques that are associated with a higher morbidity rate and many other undesirable side effects. Although there are some functional treatment methods as mentioned, there exists a need for more effective procedures using minimally invasive surgical techniques.

### BRIEF SUMMARY

Embodiments of the present invention may include a rivet introduction means and a method of using the same.

In one aspect, the invention may include a rivet introduction device. The rivet introduction device may include an elongate outer sheath having an outer sheath lumen extending therethrough, an elongate inner sheath having an inner sheath lumen extending therethrough, and an elongate mandrel having a wire guide lumen extending therethrough. The inner sheath may be disposed in a longitudinally slidable manner through at least a portion of the outer sheath lumen, and the mandrel may be disposed in a longitudinally slidable manner through at least a portion of the inner sheath lumen. A distal end portion of the mandrel may be expandable.

In another aspect, the present invention may include a blind rivet system including at least one blind rivet means configured for securing a plurality of soft tissue surfaces together and a means for introducing and deploying the blind rivet.

In yet another aspect, the present invention may include a method of connecting soft tissue surfaces. The method may include the steps of: directing a penetrating means through a first tissue surface and a second tissue surface to create an opening therethrough; directing a wire guide through the opening; providing the rivet introduction device of the present invention; directing the rivet introduction device over the wire guide to the opening, the wire guide being disposed through the wire guide lumen such that the distal end portion of the mandrel is expanded to an outer diameter that is greater than the inner diameter of the rivet lumen; directing a distal portion of the mandrel and a distal portion of the rivet means through the opening such that the first expansion zone is disposed distal of the opening relative to the rivet introduction device; and holding the rivet means in place with the inner and outer sheaths while drawing the mandrel proximally such that the distal end portion of the mandrel contacts a distal portion of the rivet means with sufficient force to expand the first expansion zone.

### BRIEF DESCRIPTION OF THE DRAWINGS

FIG. 1A is a perspective view of an unexpanded first rivet embodiment;

FIG. 1B depicts a perspective view of the rivet embodiment of FIG. 1A in a single-expanded state;

FIG. 1C shows a perspective view of the rivet embodiment of FIG. 1A in a dual-expanded state;

FIG. 2A is an exterior view of a rivet introduction device embodiment;

FIG. 2B illustrates a longitudinal section view of the rivet introduction device of FIG. 2A;

FIGS. 3A-3F illustrate a method of using the rivet introduction device;

FIGS. 4A-4C show another rivet embodiment; and

FIGS. 5A-5D show still another rivet embodiment.

### DETAILED DESCRIPTION

In one aspect, the present invention includes a rivet introduction device configured for use with an endoscope such as, for example, a duodenoscope. Those of skill in the art will appreciate that embodiments of the present invention may also be used with other minimally invasive surgical devices and systems such as, for example, a laparoscopy system including a laparoscope and one or more accessory trocars. Embodiments of the present invention may present advantages of ease of use and mechanical simplicity as compared to endoscopic suturing devices and other presently-existing devices configured for repairing or otherwise connecting tissue.

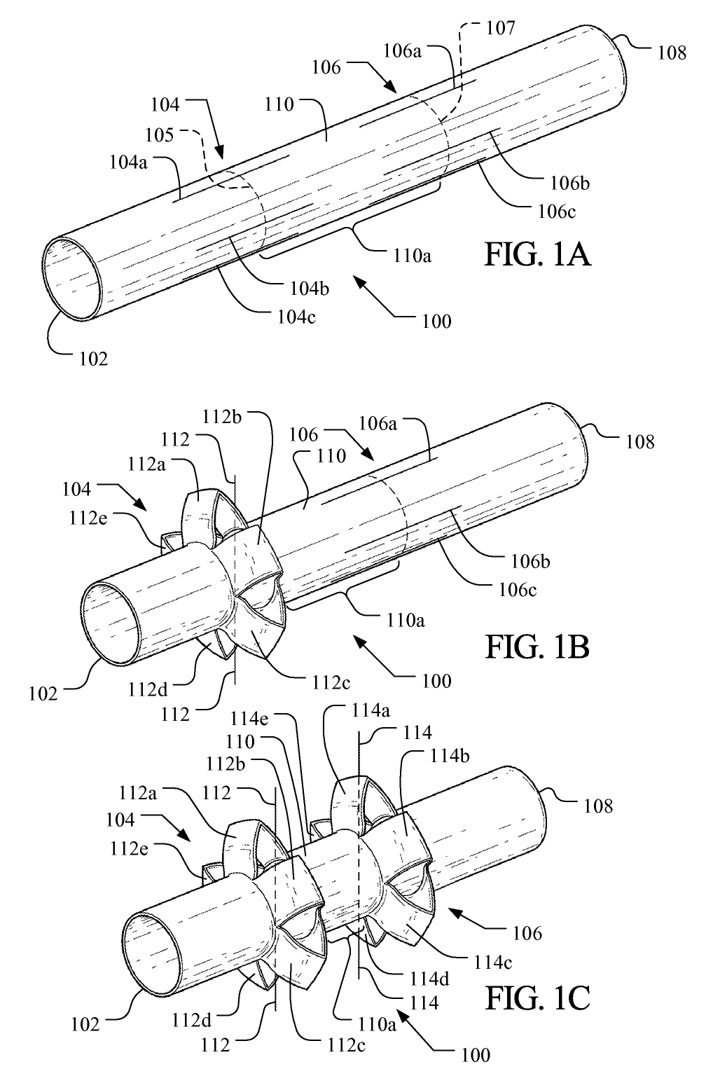

In embodiments of the present invention different rivet embodiments may be used with a rivet introduction device in a rivet introduction system. FIGS. 1A-1C show one embodiment of a rivet 100. The rivet 100 is configured as a blind rivet, by which it is meant that a user does not need direct access to a distal rivet end 102 to deploy first and second expansion zones 104, 106 of the rivet 100. In unexpanded form, the rivet 100 may be a generally cylindrical column with a central longitudinal rivet lumen (although those of skill in the art will appreciate that other geometries may be used within the scope of the present invention). As shown in FIG. 1A, the rivet 100 includes an elongate body portion 110 having a proximal rivet end 108 and a distal rivet end 102. The rivet 100 may be constructed of any material having appropriate rigidity and malleability qualities including, for example, a polymer, nitinol, or another alloy such as, for example, 304 stainless steel; further, the rivet may be constructed of any combination thereof such as, for example, a composite of plastic and metal (e.g., metal core coated by plastic, plastic capture zone with metal expansion regions or vice versa). The rivet 100 may also include visualization markers such as radio-opaque, luminescent, or other markings that will allow a user to visualize the rivet 100. The elongate body portion 110 includes the first expansion zone 104 and the second expansion zone 106, with a tissue capture zone 110a therebetween. Each of the expansion zones 104, 106 includes a series of apertures such as, for example, a series of slits 104a-n, 106a-n through the rivet wall (where “n” represents a letter corresponding to a highest-numbered aperture). In the illustrated embodiment, the series of slits 104a-n, 106a-n are generally parallel, but parallel structure is not required. As shown in FIG. 1A, each expansion zone 104, 106 may include a central region 105, 107 that is pre-stressed or otherwise configured to deform more readily than adjacent regions of the respective expansion zone.

FIG. 1B depicts a single-expanded configuration of the rivet 100, wherein the first expansion zone 104 has been expanded to form a first securement plane 112, which is disposed generally transverse to the longitudinal axis of the elongate body portion 110 along a line 112-112. The first securement plane 112 includes a series of petals 112a-n that have been folded outward between the series of slits 104a-n by a longitudinal compression of the elongate body portion 110 of the rivet 100. FIG. 1C depicts a double-expanded configuration of the rivet 100, wherein in addition to the first expansion zone 104 having been expanded, the second expansion zone 106 has also been expanded to form a second securement plane 114, which is disposed generally transverse to the longitudinal axis of the elongate body portion 110 along a line 114-114. The second securement plane 114 includes a series of petals 114a-n that have been folded outward between the series of slits 106a-n by a longitudinal compression of the elongate body portion 110 of the rivet 100. In some embodiments, the regions between the series of slits 104a-n, 106a-n may be pre-stressed such that they more readily deform to form the series of petals 112a-n, 114a-n.

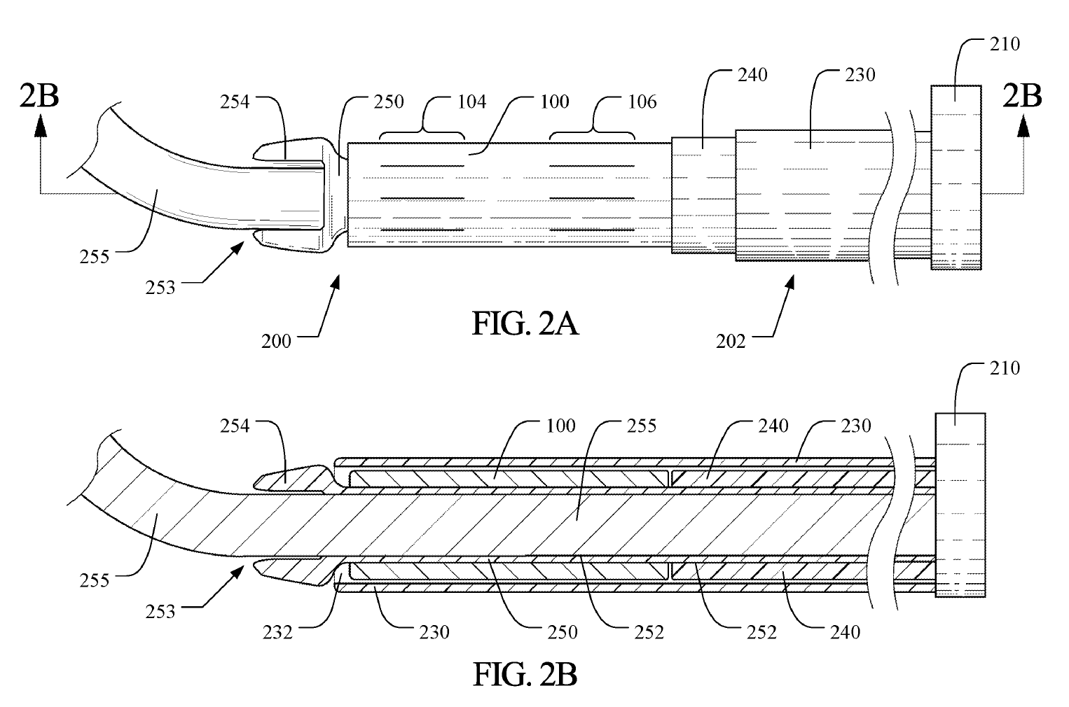

FIG. 2A illustrates a rivet introduction system 200 of the present invention, including a rivet introduction device 202 and a rivet 100 (of the type shown in FIGS. 1A-1C), with the components extended for visibility. FIG. 2B shows a longitudinal cross-section of the components in a non-extended configuration, such as might be used when introducing the rivet introduction system 200 through the working channel of an endoscope or through a trocar of a laparoscopy system. The rivet introduction device 202 includes a proximal handle 210 (shown only diagrammatically; those of skill in the art will appreciate that a standard or modified three-ring handle or other handle configuration currently known or developed in the future may be used within the scope of the present invention). An outer sheath 230 extends distally from the proximal handle 210 and is coaxially disposed around an inner sheath 240, which is longitudinally slidable through an outer sheath lumen 232. In the illustrated embodiment, the inner sheath 240 has about the same outer diameter as the rivet 100. The inner sheath 240 includes an inner sheath lumen 242 through which a mandrel 250 extends longitudinally. The mandrel 250 includes a wire guide lumen 252, through which a wire guide 255 is disposed.

A distal portion of the mandrel 250 includes a flexible mouth portion 253 having jaw members 254. As is also shown in FIG. 2A, when the wire guide 255 is extended between the jaw members 254, the jaw members 254 are spread apart such that the outer diameter of the flexible mouth portion 253 is greater than the inner diameter of the rivet 100. This configuration allows the mandrel 250 to hold the rivet 100 longitudinally against the inner sheath 240. There may be as few as two jaw members 254, but those of skill in the art will appreciate that a mandrel 250 may be configured to include three or more jaw members 254 that can open/close radially around a wire guide 255/ wire guide lumen mouth.

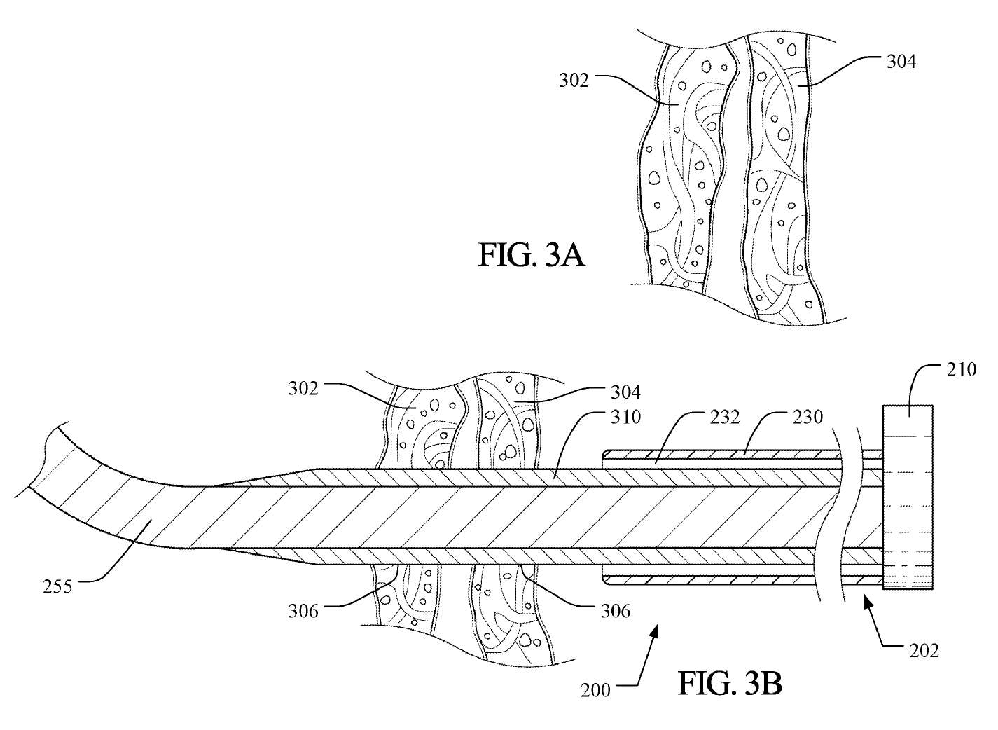

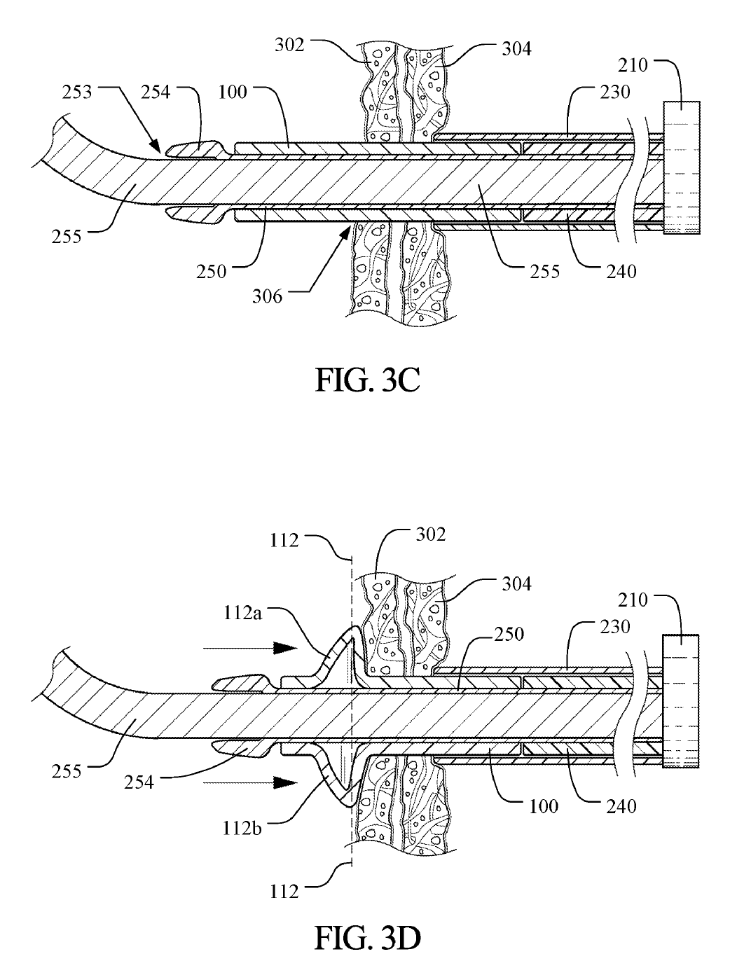

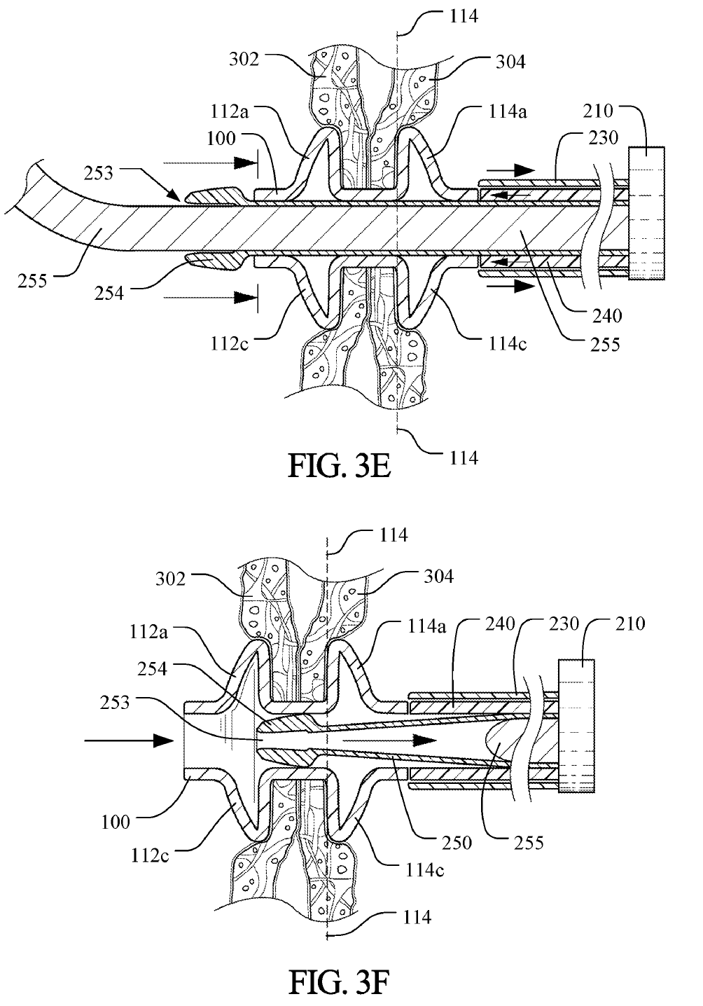

FIGS. 3A-3F illustrate a method of introducing a rivet 100 using the rivet introduction device 202 with reference to components shown in FIGS. 1A-2B. FIG. 3A shows first and second soft tissue structures 302, 304 to be connected. Those of skill in the art will appreciate that the presently described method will be applicable to a variety of tissue types and tissue-affixation settings such as, for example, repairing a tissue tear or lesion (e.g., transmural gastric lesion) or creating an anastomosis. Those of skill in the art will also appreciate that this method can be used in circumstances where only one side of tissues to be joined is accessible. As shown in FIG. 3B, a penetrating member 310 such as, for example, a “hot FNA needle” (a fine needle aspiration needle transmitting an electrical current for cutting and/or coagulation) or another appropriate needle may be directed through the first and second soft tissue structures 302, 304 to create an aperture 306 with the wire guide 255 introduced therethrough. The penetrating member 310 may be withdrawn, and the rivet introduction system 200 may be directed along the wire guide 255 to the tissue site to be treated.

Then, as shown in FIG. 3C, the mandrel 250, together with the rivet 100 around the mandrel 250, may be directed through the aperture 306 such that the first expansion zone 104 (see FIG. 2A) is distal of the aperture 306. Next, as shown in FIG. 3D, the jaw members 254 of the mandrel 250 may be drawn proximally against the distal rivet end 102 with sufficient force to expand the series of petals 112a-n of the first expansion zone 104 to form the first securement plane 112 on the distal side of the first soft tissue structure 302. The distal end of the inner sheath 240 preferably prevents proximal movement of the rivet 100 such that the proximally-directed force of the mandrel 250 against the rivet 100 will expand the first expansion zone 104 rather than moving the rivet 100 proximally. The outer sheath 230 remains disposed around the second expansion zone 106 (see FIG. 2A), preventing it from expanding. The wire guide 255 is disposed through the flexible mouth portion 253 of the mandrel 250, keeping the flexible mouth portion 253 expanded so that the jaw members 254 contact the distal end of the rivet 100 with sufficient force to expand the first expansion zone 104. At this point, the entire assembly may be pulled proximally to position the first and second soft tissue structures 302, 304 in close contact.

Next, as shown in FIG. 3E, the outer sheath 230 may be drawn proximally such that the second expansion zone 106 is exposed. Then, the mandrel 250 may be held stationary against the distal rivet end 102, while the inner sheath 240 is advanced distally with sufficient force to expand the series of petals 114a-n of the second expansion zone 106 to form the second securement plane 114 on the proximal side of the second soft tissue structure 304. Alternatively, the mandrel 250 may be drawn proximally, while the distal end of the inner sheath 240 preferably prevents proximal movement of the rivet 100 such that the proximally-directed force of the mandrel 250 against the rivet 100 will expand the second expansion zone 106 rather than move the rivet 100 proximally. In either of these manners, the first and second securement planes 112, 114 may secure the first and second soft tissue structures 302, 304 together.

After the first and second soft tissue structures 302, 304 are secured, the rivet introduction device 202 may be withdrawn. FIG. 3F shows that the wire guide 255 may be withdrawn to a location proximal of the flexible mouth portion 253 of the mandrel 250. Then, the mandrel 250 may be withdrawn through the rivet 100 as—without the wire guide 255 present to keep the flexible mouth portion 253 open—the outer diameter of the flexible mouth portion 253 can be collapsed to fit through the inner diameter of the rivet 100. As a final step, the rivet introduction device 202 may be withdrawn away from the rivet 100. Those of skill in the art will appreciate that visualization of the steps of this method may be accomplished by direct visualization using a camera or other viewing instrumentality of an endoscope, ultrasound, fluoroscopy, and/or a combination thereof. In a preferred embodiment of the method, the size of the rivet 100 and its relative dimensions (e.g. lengths of tissue capture zone and expansion zones) are selected based upon the dimensions and type(s) of the tissue(s) to be secured.

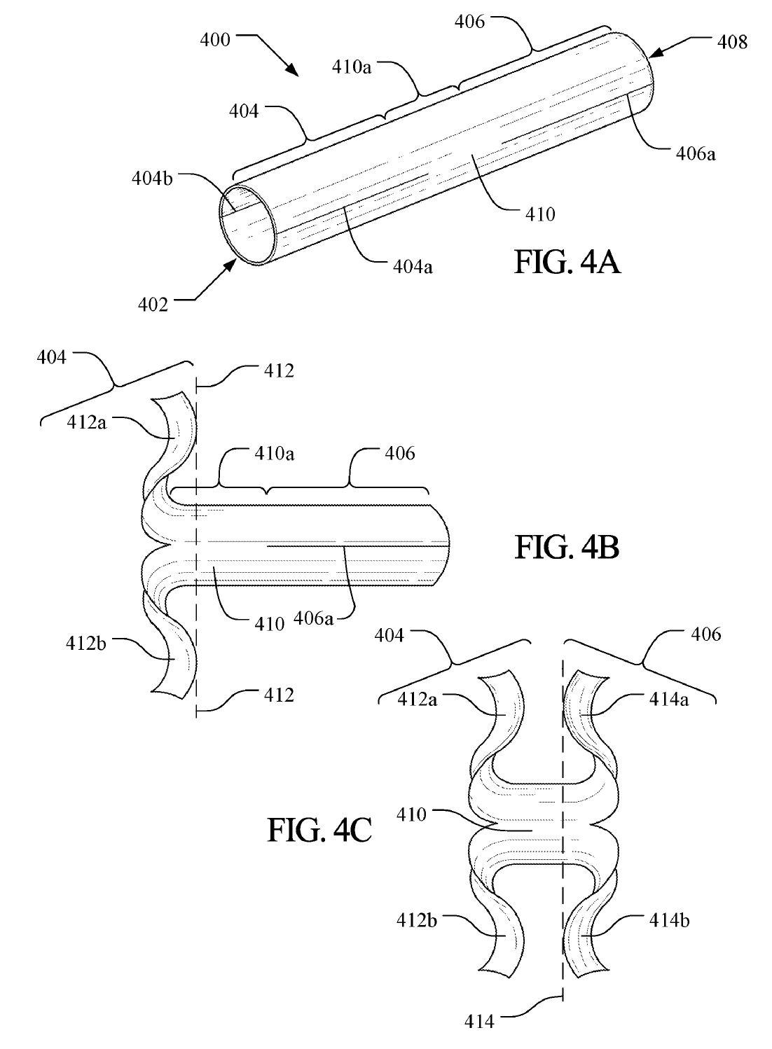

FIGS. 4A-4C show another embodiment of a rivet 400. The rivet 400 is configured as a blind rivet, by which it is meant that a user does not need direct access to a distal rivet end 402 to deploy first and second expansion zones 404, 406 of the rivet 400. In unexpanded form, the rivet 400 may be embodied as a generally cylindrical column with a central longitudinal rivet lumen (although those of skill in the art will appreciate that other geometries may be used within the scope of the present invention). As shown in FIG. 4A, the rivet 400 includes an elongate body portion 410 having a proximal rivet end 408 and a distal rivet end 402. The rivet 400 may be constructed of any material having appropriate rigidity and malleability qualities including, for example, a polymer, stainless steel, or another alloy. The rivet 400 may also include visualization markers such as radio-opaque, luminescent, or other markings that will allow a user to visualize the rivet 400. The elongate body portion 410 includes the first expansion zone 404 and the second expansion zone 406, with a tissue capture zone 410a therebetween. Each of the expansion zones 404, 406 includes a series of apertures such as, for example, a series of slits 404a-n, 406a-n through the rivet wall (where “n” represents a letter corresponding to a highest-numbered aperture). In the illustrated embodiment, the series of slits 404a-n, 406a-n are generally parallel, but parallel structure is not required.

FIG. 4B depicts a single-expanded configuration of the rivet 400, wherein the first expansion zone 404 has been expanded to form a first securement plane 412, which is disposed generally transverse to the longitudinal axis of the elongate body portion 410 along a line 412-412. The first securement plane 412 includes a series of petals 412a-n that have been folded outward between the series of slits 404a-n by a longitudinal compression of the elongate body portion 410 of the rivet 400 (where, for 412a-n, “n” represents a letter corresponding to a highest-numbered petal). FIG. 4C depicts a double-expanded configuration of the rivet 400, wherein in addition to the first expansion zone 404 having been expanded, the second expansion zone 406 has also been expanded to form a second securement plane 414, which is disposed generally transverse to the longitudinal axis of the elongate body portion 410 along a line 414-414. The second securement plane 414 includes series of petals 414a-n that have been folded outward between the series of slits 406a-n by a longitudinal compression of the elongate body portion 410 of the rivet 400. In some embodiments, the regions between the series of slits 404a-n, 406a-n may be pre-stressed such that they more readily deform to form the series of petals 412a-n, 414a-n.

In one embodiment, the rivet 400 may be introduced and deployed in the same manner as illustrated with reference to FIGS. 3A-3F, using a mandrel 250 to expand the petals 412a-n, 414a-n. In another embodiment, the series of petals 412a-n, 414a-n may be constructed of a memory material such as a polymer and/or alloy that is biased into an expanded configuration. In a method for deploying a memory material embodiment of a rivet 400 using a rivet introduction device 202, the outer sheath 230 is kept around the first expansion zone 404 until it is in a position to be expanded (e.g., adjacent a distal surface of tissue being secured). Then the outer sheath 230 may be withdrawn, allowing the series of petals 412a-n of the distal first expansion zone 404 to open. Similarly, the second expansion zone 406 can be allowed to open by further retracting the outer sheath 230 when the second expansion zone 406 is in a desired position (e.g., adjacent a distal surface of tissue being secured), such that the series of petals 414a-n of the second expansion zone 406 are allowed to open.

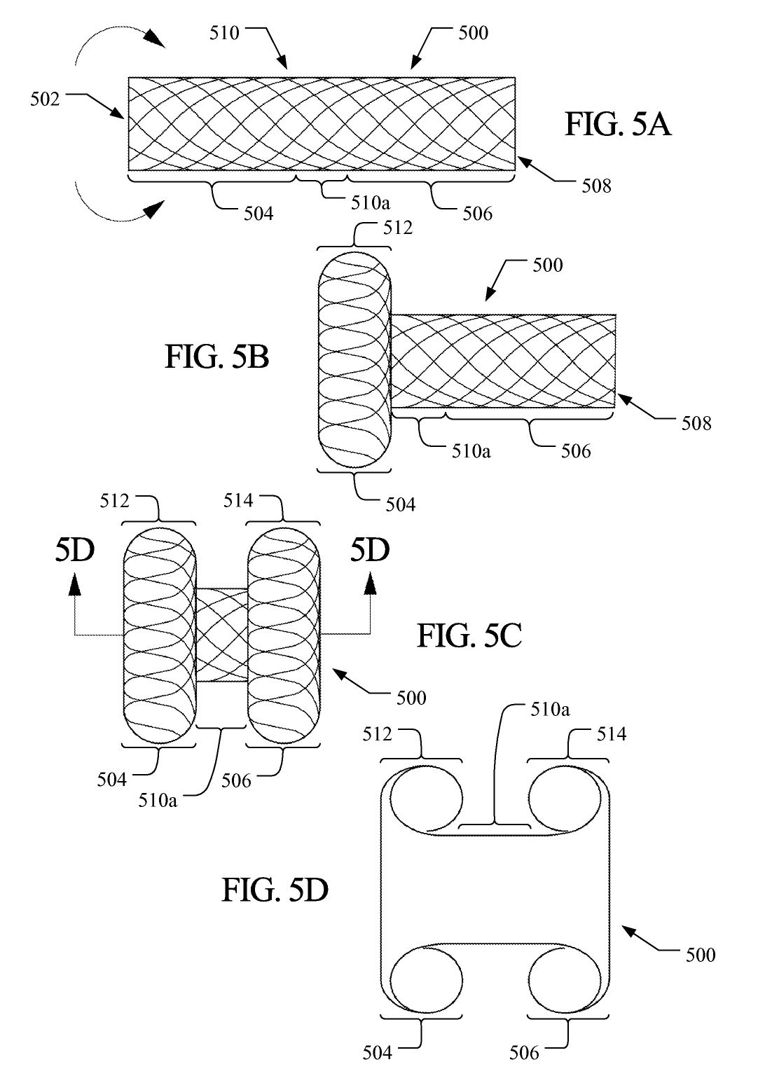

FIGS. 5A-5C show yet another embodiment of a rivet 500. The rivet 500 is configured as a blind rivet, by which it is meant that a user does not need direct access to a distal rivet end 502 to deploy first and second expansion zones 504, 506 of the rivet 500. In unexpanded form, the rivet 500 may be embodied as a generally cylindrical column constructed of a mesh or other wire arrangement similar to an open-sided stent with a central longitudinal rivet lumen (although those of skill in the art will appreciate that other geometries may be used within the scope of the present invention). In one embodiment, the rivet 500 may be constructed of a memory material or another material biased into the shape described below and shown in FIGS. 5A-5C, such that a deployment thereof does not require a mandrel. Additionally, the rivet 500 may be deployed in the manner described above (with reference to a memory material embodiment of the rivet 400).

As shown in FIG. 5A, the rivet 500 includes an elongate body portion 510 having a proximal rivet end 508 and a distal rivet end 502. The rivet 500 may be constructed of any material having appropriate rigidity and malleability qualities including, for example, a polymer, nitinol, or another alloy. The rivet 500 may also include visualization markers such as radio-opaque, luminescent, or other markings that will allow a user to visualize the rivet 500. The elongate body portion 510 includes the first expansion zone 504 and the second expansion zone 506, with a tissue capture zone 510a therebetween. Each of the expansion zones 504, 506 is pre-formed or otherwise biased to “roll” into an open configuration (similar to, for example, the rolling action used in rolling down socks on a person's leg) using a memory material such as, for example, nitinol. During an application of the rivet 500, the rivet 500 is held in its unexpanded configuration by an outer sleeve (not shown) that may be withdrawn when the rivet 500 is in a desired position, in order to allow expansion of the first and second expansion zones 504, 506.

FIG. 5B depicts a single-expanded configuration of the rivet 500, wherein the first expansion zone 504 has been expanded to form a first securement region 512. FIG. 5C depicts a double-expanded configuration of the rivet 500, wherein in addition to the first expansion zone 504 having been expanded, the second expansion zone 506 has also been expanded to form a second securement region 514. In this embodiment, the first and second expansion zones 504, 506 are biased into an open configuration, but they may alternatively be configured to be rolled outward by a longitudinal compression of the elongate body portion 510 of the rivet 500 to expand to the position shown in FIG. 5C. FIG. 5D shows a longitudinal cross-section of FIG. 5C taken along a line 5D-5D, in diagrammatic fashion. As shown therein, the first and second expansion zones 504, 506 are “curled open” or “rolled open” on either side of the tissue capture zone 510a.

Those of skill in the art will appreciate that, in a method of applying a rivet 500, a central mandrel is not needed to expand the rivet. A central mandrel may be used to retain the rivet in a delivery sheath until desired deployment/expansion, and an outer sheath may be used to restrain the rivet 500 from deploying/expanding until it is in a desired position. Thus, deployment of a rivet 500 may be effected in a manner similar to that shown in FIGS. 3A-3F, except that the mandrel 250 plays a longitudinal retaining role, and expansion is accomplished by releasing the first or second expansion zone 504, 506 from the outer sheath 230—either by retracting the outer sheath 230, or using the inner sheath 240 to advance a portion of the rivet 500 distally past the distal end of the outer sheath 230 where the rivet 500 is able to open into its biased-open shape to form the first or second securement region 512, 514.

Those of skill in the art will appreciate that the different petal/expansion zone configurations shown and described may be combined with each other or with other petal/expansion zone embodiments within the scope of the present invention. Those of skill in the art will appreciate that other embodiments of the rivet introduction device and system described herein may also be practiced within the scope of the present application. It is therefore intended that the foregoing detailed description be regarded as illustrative rather than limiting. It should be understood that the following claims, including all equivalents, are intended to define the spirit and scope of this invention.

We claim:

1. A medical device configured for introducing a blind rivet through soft tissue, the device comprising:
a rivet introduction device comprising:
an elongate outer sheath having an outer sheath lumen extending therethrough;
an elongate inner sheath having an inner sheath lumen extending therethrough; and
an elongate mandrel having a wire guide lumen extending therethrough;
wherein the elongate inner sheath is disposed in a longitudinally slidable manner through at least a portion of the outer sheath lumen and the elongate mandrel is disposed in a longitudinally slidable manner through at least a portion of the inner sheath lumen;
wherein a distal end portion of the elongate mandrel includes flexible structure configured to allow radial expansion/contraction; and
a rivet disposed coaxially around the elongate mandrel adjacent the distal end portion of the elongate mandrel.

2. The medical device of claim 1, wherein the rivet further comprises a rivet lumen having an inner diameter, and wherein an outer diameter of the rivet is greater than an inner diameter of the inner sheath lumen.

3. The medical device of claim 2, further comprising a wire guide extending through the wire guide lumen of the mandrel.

4. The medical device of claim 3, wherein the wire guide extends through the distal end portion of the elongate mandrel such that an outer diameter of the distal end portion of the elongate mandrel is expanded to be larger than an inner diameter of the rivet lumen.

5. The medical device of claim 2, wherein the rivet comprises first and second expansion zones.

6. The medical device of claim 5, wherein the rivet comprises a generally cylindrical shape with a rivet wall surrounding the rivet lumen, the rivet lumen open at a proximal end and a distal end of the rivet.

7. The medical device of claim 6, wherein one of the first and second expansion zones of the rivet comprises a series of generally parallel apertures through the rivet wall nearer the distal end of the rivet, and the other of the first and second expansion zones comprises a series of generally parallel apertures through the rivet wall nearer the proximal end of the rivet.

8. The medical device of claim 7, wherein at least one of the generally parallel apertures of the rivet extends to the proximal end of the rivet.

9. The medical device of claim 7, wherein at least one of the generally parallel apertures of the rivet extends to the distal end of the rivet.

10. The medical device of claim 6, wherein each of the first and second expansion zones is configured to expand to an outer diameter that is greater than an outer diameter of the rivet wall upon application of a longitudinal force to the rivet.

11. The medical device of claim 1, wherein the distal end portion of the elongate mandrel comprises a plurality of separated jaw structures.

12. The medical device of claim 5, wherein at least one of the first and second expansion zones of the rivet is biased into an expanded configuration.

13. The medical device of claim 12, wherein the at least one of the first and second expansion zones of the rivet assumes the expanded configuration when not circumferentially restrained by the elongate outer sheath.

14. A medical blind rivet system comprising:
at least one blind rivet means configured for securing a plurality of soft tissue surfaces together;
a means for introducing and deploying the blind rivet means;
wherein the at least one blind rivet means comprises first and second expansion zones on either side of a central region having a first diameter; and
wherein each of the first and second expansion zones is configured to be expandable to a diameter that is greater than the first diameter.

15. The medical blind rivet system of claim 14, wherein the at least one blind rivet means comprises a generally cylindrical rivet wall defining a rivet lumen, the rivet lumen being open at a proximal end and distal end of the blind rivet means.

16. The medical blind rivet system of claim 15, wherein at least one of the first and second expansion zones comprises a plurality of apertures through the generally cylindrical rivet wall.

17. The medical blind rivet system of claim 16, wherein the plurality of apertures through the generally cylindrical rivet wall define a plurality of petal-like structures configured to spread apart from each other.

18. The medical blind rivet system of claim 17, wherein the plurality of petal-like structures are configured to form a generally planar structure spread apart from each other, the planar structure being generally perpendicular to a longitudinal axis of the generally cylindrical rivet wall.

19. The medical blind rivet system of claim 15, wherein the means for introducing and deploying the blind rivet means comprises:
an elongate outer sheath defining an outer sheath lumen that extends through at least a longitudinal portion of the elongate outer sheath;
an elongate inner sheath defining an inner sheath lumen that extends through at least a longitudinal portion of the elongate inner sheath; and
an elongate mandrel having a wire guide lumen that extends through at least a longitudinal portion of the elongate mandrel;
wherein the elongate inner sheath is disposed in a longitudinally slidable manner through at least a portion of the outer sheath lumen and the elongate mandrel is disposed in a longitudinally slidable manner through at least a portion of the inner sheath lumen;
wherein a distal end portion of the elongate mandrel is expandable to an outer diameter that is greater than an inner diameter of the rivet lumen;
and wherein the blind rivet means is disposed about a distal portion of the elongate mandrel.

20. A method of connecting tissue surfaces, the method comprising the steps of:
directing a penetrating means through a first tissue surface and a second tissue surface to create an opening therethrough;
directing a wire guide through the opening;
providing the medical blind rivet system of claim 19;
directing the medical blind rivet system over the wire guide to the opening, the wire guide being disposed through the wire guide lumen such that the distal end portion of the elongate mandrel is expanded to an outer diameter that is greater than the inner diameter of the rivet lumen;
directing a distal portion of the elongate mandrel and a distal portion of the blind rivet means through the opening such that the first expansion zone is disposed distal of the opening relative to the medical blind rivet system; and
holding the blind rivet means in place with the elongate inner and outer sheaths while drawing the elongate mandrel proximally such that the distal end portion of the elongate mandrel contacts a distal portion of the blind rivet means with sufficient force to expand the first expansion zone.

21. The method of claim 20, further comprising the steps of:
moving the medical blind rivet system such that the second expansion zone of the blind rivet means is proximal of the opening;
moving one of the elongate outer sheath and elongate inner sheath relative to the other such that the elongate outer sheath does not cover the second expansion zone; and
holding the blind rivet means in place with the elongate inner sheath while drawing the elongate mandrel proximally such that the distal end portion of the elongate mandrel contacts a distal portion of the blind rivet means with sufficient force to expand the second expansion zone.
